(19) United States
(12) Patent Application Publication
(10) Pub. No.: US 2013/0053277 A1
(43) Pub. Date: Feb. 28, 2013

(54) METHODS OF IDENTIFYING INHIBITORS OF POLYPEPTIDES-OF-INTEREST

(71) Applicant: Isaiah Arkin, Har-Adar (IL)

(72) Inventor: Isaiah Arkin, Har-Adar (IL)

(73) Assignee: Yissum Research Development Company of the Hebrew University of Jerusalem Ltd., Jerusalem (IL)

(21) Appl. No.: 13/696,110

(22) PCT Filed: May 4, 2011
(86) PCT No.: PCT/IL11/00354

§ 371 Date: Nov. 5, 2012

Related U.S. Application Data
(60) Provisional application No. 61/331,152, filed on May 4, 2010.

(51) Int. Cl. G01N 33/566; C12N 1/21; C40B 30/06; G01N 33/573; C12Q 1/68

(52) U.S. Cl. 506/10; 435/7.32; 435/7.8; 435/6.15; 435/252.3

(57) ABSTRACT

Methods of identifying inhibitors of polypeptide-of-interests are provided. Accordingly there is provided a method comprising:
(a) expressing the polypeptide-of-interest in cells being xenogeneic to the polypeptide, wherein the polypeptide-of-interest is selected causing growth retardation of the cells when expressed therein;
(b) contacting the cells expressing the polypeptide-of-interest with a test agent;
(c) measuring growth of the cells following or concomitant with step (b), wherein a relief in the growth retardation is indicative that the test agent is an inhibitor of the polypeptide-of-interest; alternatively or additionally, the method is performed by:
(a) expressing the polypeptide-of-interest in cells being xenogeneic to the polypeptide, wherein the polypeptide-of-interest is selected causing growth retardation of the cells when expressed therein;
(b) culturing the cells expressing the polypeptide-of-interest under conditions which relieve the growth retardation, wherein the relief of the growth retardation is indicative of conditions that inhibit the polypeptide-of-interest.

### RELATED APPLICATION/S

This Application claims priority from U.S. Provisional Patent Application No. 61/331,152 filed on May 4, 2010, the contents of which are hereby incorporated by reference in its entirety.

### FIELD AND BACKGROUND OF THE INVENTION

The present invention, in some embodiments thereof, relates to methods of identifying inhibitors of polypeptides-of-interest.

The embodiment of Paul Ehrlich's “magic bullet” vision is a chemical capable of specifically inhibiting the function of a harmful pathogen. Alternatively, a chemical that specifically alters one of the body's pathological processes. The fulfillment of this vision has shaped the way in which the pharmaceutical industry operates. More specifically, the search for potent compounds is the major thrust of the pharmaceutical industry.

Towards this end a general approach has been developed to identify such chemicals, which is not unlike Ehrlich's own search for an anti trypanosome agent (trypan red). The process involves three components:

The assay: The most important aspect of any research aimed at developing a drug against a harmful microorganism, or an aberrant bodily process, is the development of a suitable assay. The assay should enable rapid testing of a vital function of the organism or of the aberrant process. The efficacy of any compound can then be directly tested, by adding it to the assay mixture to see if it inhibits the particular functionality.

The compound library: What agents one can add depends on the proprietary compound library, which in many pharmaceutical companies can be as large as several hundreds of thousands of chemicals. A significant part of these libraries is taken up by natural products made by microorganisms, normally termed secondary metabolites. If shown to inhibit the growth of any microorganism they are classified as antibiotics.

Screening technology: High throughput automation technology enables one to screen a very large number of compounds, testing their ability to inhibit the particular function assayed. This is where the nature of the assay is critical in lending itself to high throughput screening of a large number of compounds as possible.

Eventually, in a completely fortuitous process, a lead compound may be identified that inhibits the particular functionality assayed.

Numerous approaches have been suggested attempting to employ the above paradigm in the search for ultimate drug leads.

For Example:

WO02/18537 teaches screening of mammalian cells having an abnormal mammalian cellular phenotype for agents that reverse that phenotype, while employing imaging device that structurally monitors the cellular phenotype. The method is also taught in the context of high throughput screening.

U.S. Patent Application 20040106154 teaches a drug screening assay comprising providing a cell that expresses a pair of fusion proteins which upon dimerization activate a cellular readout: providing a first compound and a second compound, each being capable of binding to one of the pair of fusion proteins, the first and second compound comprising a portion through which the first and second compounds are coupled by the action of the bond forming protein to be screened; and screening for the cellular readout, wherein a change in the cellular readout indicates catalysis of bond formation by the protein to be screened.

U.S. Patent Application numbers 20020164602, 20080026389 and 20050239157 each teaches screening for antibacterial agents using bacterial cells.

Kleymann and Werling 2004 The society for Biomolecular Screening (578-587) teach a high throughput screening assay to identify, evaluate and optimize agents for drug therapy. Host cells are infected with microbes and incubated in the presence of the test sample. Cell survival is monitored.

### SUMMARY OF THE INVENTION

According to an aspect of some embodiments of the present invention there is provided a method of identifying an inhibitor of a polypeptide-of-interest, the method comprising:

(a) expressing the polypeptide-of-interest in cells being xenogeneic to the polypeptide, wherein said polypeptide-of-interest is selected causing growth retardation of said cells when expressed therein;

(b) contacting said cells expressing said polypeptide-of-interest with a test agent;

(c) measuring growth of said cells following or concomitant with step (b), wherein a relief in said growth retardation is indicative that said test agent is an inhibitor of said polypeptide-of-interest.

According to some embodiments of the invention, the method further comprising synthesizing the test agent being the inhibitor of said polypeptide-of-interest.

According to an aspect of some embodiments of the present invention there is provided a method of identifying an inhibitor of a polypeptide-of-interest, the method comprising:

(a) expressing the polypeptide-of-interest in cells being xenogeneic to the polypeptide, wherein said polypeptide-of-interest is selected causing growth retardation of said cells when expressed therein;

(b) culturing said cells expressing said polypeptide-of-interest under conditions which relieve said growth retardation, wherein said relief of said growth retardation is indicative of conditions that inhibit said polypeptide-of-interest.

According to an aspect of some embodiments of the present invention there is provided a method of identifying a polypeptide which is incompatible with cell growth or vitality, the method comprising: (a) expressing the polypeptide in cells being xenogeneic to the polypeptide; and

(b) measuring growth of said cells expressing the polypeptide compared to growth of control cells of the same origin not expressing the polypeptide under identical conditions, wherein when growth of said cells expressing the polypeptide is retarded compared to said control cells, the polypeptide is considered incompatible with cell growth or vitality.

According to some embodiments of the invention, said conditions comprise a molecule endogenously synthesized by said cells.

According to some embodiments of the invention, the method further comprising synthesizing said molecule.

According to some embodiments of the invention, further comprising contacting said cells expressing said polypeptide-of-interest with said molecule to identify said relief of said growth retardation, thereby validating said inhibitory activity of said molecule.

According to some embodiments of the invention, said polypeptide-of-interest is a recombinant peptide.

According to some embodiments of the invention, a selection of said polypeptide of-interest causing growth retardation of said cell culture is performed according to the method of claim 1.

According to some embodiments of the invention, wherein said cells are microbial cells.

According to some embodiments of the invention, microbial cells are bacterial cells.

According to some embodiments of the invention, said bacterial cells comprise Gram positive bacteria.

According to some embodiments of the invention, said bacterial cells comprise Gram negative bacteria.

According to some embodiments of the invention, said polypeptide is a human polypeptide.

According to some embodiments of the invention, expressing the polypeptide comprises induced expression.

According to some embodiments of the invention, said expressing is effected at least in part in a presence of a known inhibitor of said polypeptide so as to prevent death of said cells.

According to some embodiments of the invention, said polypeptide is a disease causing polypeptide.

According to some embodiments of the invention, said polypeptide-of-interest is selected from the group consisting of an ion channel and a protease.

According to some embodiments of the invention, said test agent comprises a nucleic acid sequence and wherein contacting refers to transforming said cells to express said nucleic acid sequence.

According to some embodiments of the invention, is effected in high throughput configuration.

According to some embodiments of the invention, said test agent forms a part of a library.

According to some embodiments of the invention, said disease causing polypeptide comprises a viral polypeptide.

According to some embodiments of the invention, said viral polypeptide comprises an influenza polypeptide.

According to some embodiments of the invention, said influenza polypeptide is M2.

According to some embodiments of the invention, there is provided an isolated bacterial cell expressing an influenza polypeptide.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. [IF IMAGES, REPHRASE] With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a Western blot analysis of the ma1E-M2 chimeric protein as a function of time post induction by IPTG 50 μM, and the presence of the anti-flu channel blocker rimantadine at 100 μM. Bacterial cells were examined by sodium dodecyl sulfate polyacrylamide gel electrophoresis and blotted by an anti-His-Tag antibody. Molecular weights are shown on the left.

FIG. 2 is a graphic presentation showing growth curves of bacteria expressing the M2 channel (induced) and the influence of the anti-viral channel blocker rimantadine (in gray), thereupon. Bacteria that do not express the ion channel (uninduced) are shown as control. Induction takes place when the bacteria density reaches an O.D.600 of 0.1;

FIG. 3 is a graphic presentation showing representative growth of bacteria expressing different M2 channel variants in the presence of rimantadine (top) or amantadine (bottom), as indicated. Growth values without any drug treatment (e.g. FIG. 2, induced) are subtracted as control from the above results. The different channel variants are: Singapore—black; Rostock wt—gray; 8M2—dotted black; Singapore S31N—dotted gray and swine flu-dashed. The concentration of both drugs was 70 μM;

FIG. 4 is a graphic presentation showing dose response curves of amantadine and rimantadine for various M2 channels (as indicated) upon the growth rate of the host bacteria. Note different drug concentration for each panel. Experimental values (black diamonds) were fit according to the Monod equation (black lines) yielding the Ks values as indicated. The residuals are shown in gray squares;

FIGS. 5A-C are graphs showing bacterial cell growth influenced by the expression of Vpu and p7 in DH5-alpha (A) EP432 (B) and KNabc (C) cell. Blue: control plasmid, Red control plasmid induced, Yellow HPCp7 induced and Green HIV Vpu induced.

### DESCRIPTION OF SPECIFIC EMBODIMENTS OF THE INVENTION

The present invention, in some embodiments thereof, relates to methods of identifying inhibitors of polypeptides-of-interest.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Efficacy, specificity and tolerability are the key criteria for a successful medication in the clinic.

Therefore, a new test method to obtain selective and active lead compounds has been developed. The present inventor has realized that cell growth or vitality is a simple yet accurate proxy which can be reliably used in high throughout drug screening settings. Specifically, expression of a target molecule in irrelevant (xeno) host cells under conditions in which the target protein slows cell growth or reduces cell vitality, can be used to measure the activity of the target protein and conversely inhibition thereof and as such can be used as a platform for drug screening.

As is illustrated hereinbelow and in the Examples section which follows, the present inventor designed a cell-based assay in which the M2 influenza channel protein is expressed in bacteria causing growth retardation. The effects of channel blockers were assayed by their ability to relieve the aforementioned growth retardation. Similar systems were generated for Vpu from HIV, p7 from Hepatitis C and SH from respiratory synsytial virus (RSV).

Thus, according to an aspect of the invention there is provided a method of identifying an inhibitor of a polypeptide of interest.

According to an embodiment of the invention, the method is performed by:

(a) expressing the polypeptide-of-interest in cells being xenogeneic to the polypeptide, wherein the polypeptide-of-interest is selected causing growth retardation of the cells when expressed therein;

(b) contacting the cells expressing the polypeptide-of-interest with a test agent; and

(c) measuring growth of the cells following—or concomitant with step (b), wherein a relief in the growth retardation is indicative that the test agent is an inhibitor of the polypeptide-of-interest.

According to an alternative embodiment, the method is performed by:

(a) expressing the polypeptide-of-interest in cells being xenogeneic to the polypeptide, wherein the polypeptide-of-interest is selected causing growth retardation of said cells when expressed therein;

(b) culturing the cells expressing said polypeptide-of-interest under conditions which relieve said growth retardation, wherein the relief in the growth retardation is indicative of conditions that inhibit said polypeptide-of-interest.

While the first embodiment relies on screening of exogenously added test agents, the latter embodiment relies on the endogenous production of a molecule (e.g., a biomolecule, being a metabolite, a protein, a lipid, a carbohydrate, a nucleic acid or a combination of same) which infers resistance to the target polypeptide. Such a molecule is the inhibitory compound against the target polypeptide and is based upon forced evolution. Basically, the target protein is “made” harmful to the cells in an attempt to encourage the cells to generate a molecule that inhibits the target polypeptide. Subsequently, relying upon the uncanny ability of cultured cells in general and microorganisms in particular to develop resistance against any harmful stress, the evolution will force them to produce an inhibitor (e.g., an anti pathogen) compound. Such a compound can then be used as a therapeutic agent.

As used herein the phrase “polypeptide-of-interest”, also referred to herein as “target polypeptide” or “target protein” refers to an amino acid polymer of a length anywhere between a few or several amino acids to several thousand amino acids, which can represent either a fraction or an entire sequence of a characterized or uncharacterized protein from any source or organism. It will be appreciated that although polynucleotide fragments originating either from genomic or mRNA sources are preferably utilized to code for the polypeptides employed by the present invention, combinatorial polynucleotide sequences, which, for example, can be synthetic DNA segments of different lengths are also contemplated herein. According to a specific embodiment, the polypeptide-of-interest is not a chimeric protein but rather represents a naturally occurring protein or a fragment thereof. In certain cases however a heterologous sequence may be attached to the polypeptide such as so ensure cellular localization (e.g., membrane localization).

According to a specific embodiment, the polypeptide-of-interest is involved in the onset or progression of a medical condition (e.g., enzymes and ion channels).

According to an exemplary embodiment, the polypeptide-of-interest refers to a polypeptide or polypeptides (e.g., dimers or oligomers) that inhibition of its activity may lead to an improved clinical outcome. By one option the polypeptide-of-interest, is a target of a disease causing pathogen (virus, fungi, bacteria etc), that its inhibition may cause destruction of the pathogen or reduce infectivity of same and by this treatment of the pathogen-caused disease. By another option the disease related target is a native molecule in the subject to be treated that its inhibition may cause an improved clinical outcome (e.g., constitutively active cancer-associated tyrosine kinase receptors such as Δ2-7 EGFR associated with head and neck cancers). Other examples are provided below.

According to a specific embodiment, the polypeptide is expressed in an isolated manner that is, not forming a part of a pathogen. Accordingly the polypeptide is recombinantly expressed and therefore can be expressed from an artificial expression vector which does not include other pathogenic genes such as those necessary for infectivity.

Specific examples of polypeptides which may be used in accordance with the present teachings include, but are not limited to, enzymes, cell signaling proteins, ligand binding molecules and ion channels.

According to a specific embodiment, the polypeptide-of-interest is xenogeneic to the cell. As used herein the term “xenogeneic” refers to originating from a different species.

According to a specific embodiment, the polypeptide-of-interest shares less than 50% global identity (over the entire sequence) with any of the host cell polypeptides, and as such is not considered a functional equivalent.

Thus for instance, a eukaryotic protein is expressed in a prokaryotic system or a human pathogen is expressed in bacteria for instance. Likewise, a bacterial enzyme may be expressed in a mammalian cell.

Following is a non-limiting list of polypeptides which may be used in accordance with the present teachings.

Ion Channels

The maintenance of a membrane potential is of critical importance to all living organisms. For example, it is estimated that 75% of all of the body energy expenditure in some tissues is devoted solely to that task, and in fact to the function of a single protein, the Na⁺/K⁺ ATPase [9]. It is no surprise therefore, that agents that compromise membrane resistance to ion conduction make excellent antibiotics and are in common use throughout the animal kingdom [31, 46, 17]. The specificity of these channels is mostly determined by the delivery mechanism and not by the mode of action. In other words, the majority of pore forming agents, once inserted in the target membrane, will work perfectly well in nearly all biological membranes. This is the reverse of a specific antibiotic that inhibits a particular enzyme. It is therefore not surprising that heterologously expressed pore forming proteins are inevitably toxic to the host. It is noted that some ion channels may be inactive in their native state due to a innate regulatory component of the protein. In order for this approach to proceed this regulatory component will need to be removed to ensure that the channels are continuously active.

Thus, ion channels as a class, represent excellent targets for the present approach. Once integrated in the host's membrane in functional form they are inevitably toxic to the host. Their specific function, vital to the pathogen, makes them toxic to the expressing host (e.g., bacterial host). Finally, ion channels have long been used as highly successful targets for point intervention by pharmaceutical agents, furthering the chances of this approach succeeding.

Proteases

Specific proteases can readily be made to represent a grave threat to any bacterial host. This can be achieved by inserting a protease recognition sequence in a vital bacterial protein. Subsequent cleavage of the essential factor by the viral protease will result in lack of the essential function by the bacteria (e.g. an antibiotic resistance protein) and loss of viability. One can insert many protease recognition sequences in multiple sites increasing the probability that resistance will not be achieved through the development of an isozyme, but rather through a protease inhibitor.

Pathogenic Viruses

Viruses have always presented a grave threat to human health. As an example, the most devastating epidemic in recorded world history occurred in 1918, in which it is estimated that around 50 million people were killed. The pandemics of 1743 and 1889-1890 were nearly as disastrous as the Spanish Flu pandemic of 1918, while more recent influenza pandemics (1957 and 1968) were thankfully less severe. HIV, a more recent threat, has resulted in the deaths of several million individuals, and it is estimated that one out of a hundred adults is a carrier of the virus. Below three critical functions of viruses that can be made harmful to a bacteria are listed.

Introduction to Viral Ion Channels

As an initial step towards understanding the molecular biology that underlies the pathogenic activity of a virus, the entire viral genome is often sequenced. The research community will subsequently tend to focus on unique viral proteins, such as the spike proteins, nucleic acid polymerases and proteases. The genomes of many viruses may often contain in addition small hydrophobic (SH) proteins, including: 3A from Poliovirus [7], 6K from Semliki Forest virus [38], SH from Simian virus 5 [13], SH from Respiratory Syncytial Pneumovirus [6], M2 from Influenza A [34], BM2 from Influenza B [44,51], CM2 from Influenza C [14] and vpu from HIV [5]. M2 is by far the best characterized member of the SH protein family and exhibits properties of ion channel activity and homo-oligomerization which may be typical of the entire family. Expression of viral ion channels in a variety of hosts (e.g. Escherichia coli [11], Saccharomyces cerevisiae [23] and Xenopus laevis oocytes [4]), invariably leads to membrane permeabilization and cell death. Viral ion channels are therefore in a unique position, of being harmful to man and bacteria alike through the same means as ion channels in general.

Influenza M2, BM2 and CM2

The M2 protein from Influenza A was the last step to be elucidated in the life cycle of the Influenza virus [12]. Viral attachment and entry is carried out through the activity of the major viral spike glycoprotein HA. Membrane fusion and viral genome release occur after HA undergoes a pH dependent irreversible conformational change in the acidic endosome, but it was not clear at first why HA did not change conformation in the Golgi secretory pathway where the pH is lower than that of the cytosol. The answer to this came on identifying the pH dependent ion channel activity of M2, which negates the activity of the Golgi H⁺ ATPase [34].

M2 also participates in the virus uncoating process after viral uptake by endocytosis. The passage of H⁺s from the acidic environment of the endosomal lumen into the virion lumen (through M2) weakens the interactions between the matrix protein (M1) and the ribonucleoprotein (RNP) core, enabling the release of the viral genome into the cytoplasm [3].

The ion channel activity of M2 has been investigated in some detail and has been shown to be blocked by amantidine and BL1743 which are both potent anti-Influenza agents [45]. Amantidine resistant strains of Influenza exhibit mutations in the M2 proteins that render them insensitive to amantidine ion channel blockage. Furthermore the channel is activated by low pH, and the His residues which reside in the transmembrane segment have been shown to be essential for this function [47, 37]

M2 was shown to be a homo-tetrameric membrane protein, linked by disulfide bonds [45]. Mutation of the cysteine residues does not affect the channel activity of the protein and synthetic peptides corresponding to the transmembrane domain alone exhibit similar channel activity and amantidine sensitivity [45]. Taken together, the data suggest that tetramerization is initiated by the transmembrane domain and subsequently stabilized by cytoplasmic disulfide bonds.

For the transmembrane domain of M2 reconstituted in 1,2-dioleoylsnglycero-3-phosphocholine membranes, circular dichroism spectroscopy provided evidence that the protein homo-oligomers are predominantly a helical [45]. Highly helical content was also evident from FTIR spectra in 1,2-dimyristoylsn-glycero-3-phosphocholine membranes [20]. Both solid state NMR [18, 19] and site directed FTIR [20] were able to show that the transmembrane helices of M2 are tilted from the membrane normal by about 30°-40° and that the rotational pitch angle about the helix axis of A29 is 60°.

Interestingly, a homologue of M2 is not found in Influenza B or Influenza C, both of which contain additional proteins (BM2 and CM2, respectively) with similar structural and functional characteristics, but no sequence similarity [33]. Much less is known about the CM2 protein of Influenza C virus [15] and BM2 from Influenza B [51].

CM2 has been characterized as an integral membrane glycoprotein which forms disulfide linked dimmers and tetramers. Based on the overall topology containing a 23 residue extra cellular part, a 23 residue membrane spanning part and a 69 residue cytoplasmic tail, CM2 is assumed to be structurally similar to the Influenza A M2 protein and the Influenza B BM2 protein [33, 14].

FTIR studies (assuming a canonical helix) have shown CM2 to be highly helical and that the tilt of the helices from the membrane normal is around 15° [22]. Analysis of site directed dichroism further indicated that the rotational pitch angle of G59 and L66 is roughly 220°. BM2 has been shown to have ion channel activity in lipid bilayers [51], and similarly to M2 was shown to be oligomeric [52].

HIV vpu

The 81 residue vpu phospho-protein belongs to the auxiliary proteins of the human immunodeficiency virus type 1 (HIV1) [5, 43]. It is composed of an N terminal domain, where residues 1-5 are probably extra cellular, a 22 residues segment that spans the membrane and the hydrophilic cytoplasmic C terminal domain [25]. vpu forms homo-oligomers of at least four subunits as detected by gel electrophoresis [25]. vpu is not found in the envelope of the virus particle but is expressed in the membranes of sub cellular compartments of the infected cell [43]. The C terminal cytoplasmic domain is responsible for the degradation of one of the HIV1 co receptor molecules, CD4 [39, 40], allowing the env glycoprotein to be transported to the cell surface. The N terminal domain is responsible for virus particle release [40], but the molecular basis of these actions is unknown. It has been shown that phosphorylation of the cytoplasmic domain is essential for CD4 degradation, though it is not absolutely required for virus particle release [8, 39, 40]. Virus particle release is not specific to HIV1, since vpu is capable of enhancing the particle release of different retroviruses [10]. The transmembrane domain has been studied independently from the cytoplasmic domain. By analogy with the M2 protein of Influenza A virus [24], it has been suggested that the transmembrane part of vpu may act as an ion channel [16, 24, 40, 43]. In fact, ion channel activity for monovalent cations has been observed in Xenopus oocytes and in planar lipid bilayers [39]. Recently however, these channel activities of vpu have been questioned by a report showing that vpu expression in oocytes reduces basal membrane conductance [4]. This however, was suggested to reflect lack of expression of vpu on the cell surface.

Using NMR and FTIR spectroscopy the transmembrane segment of vpu reconstituted in a lipid bilayer is found to be predominantly a helical [49, 26, 21]. Also X-Ray reflectivity data on vpu containing monolayer indicate a helical structure [50]. Site directed dichroism analysis (assuming a canonical helix) has indicated that the tilt angle of the helices in the vpu bundle is 7° and that the rotational pitch angle of residue Val13 is 283° [21].

Viral Proteases

Many viruses encode specific proteases in their genome [32]. These proteases are often essential for the viral life cycle, evident from the fact that the protease inhibitors can serve as antiviral compounds. Due to the specificity of some of these proteases and the ability to readily express them in functional form in bacteria [27] it is straight forward to render them toxic to the host bacteria.

Pathogenic Microorganisms

There are numerous pathogenic microorganism which pose grave risk to human health. Furthermore, due to their ability to develop resistance to antibiotics (a function that is advantageously used in the proposed strategy) many pathogens that used to be under control are now a re-emerging threat (e.g. tuberculosis).

Bacterial Ion Channels

It is now a well recognized fact that many microorganisms in general and bacteria in particular encode in their genomes ion channels. For example the first structure of an ion channel is the K⁺ channel KcSA from Streptomyces lividans [35]. Every one of these channels is harmful to any bacteria in exactly the same way as any other ion channel.

Bacterial Proteases

Bacteria possess many essential proteases that are highly specific. Thus they can be rendered toxic to the cell in the same way by which viral proteases are. One such example are some of the most lethal toxins secreted by clostridia bacteria that target elements of the SNARE (component of intracellular membrane fusion) complex directly. The toxins are usually composed of 2 polypeptide chains, one of which binds to elements in the neuronal tissue and helps it to internalize, while the second polypeptide. It is an incredibly specific protease that cleaves one of the components of the SNARE complex as illustrated in Table 1.

TABLE 1
Clostridial toxins and their intracellular targets.

| | | Cleavage | |
|---|---|---|---|
| Toxin | SNARE Target | site | Cell type |
| Tetanus | synaptobrevin a | Q76F77 | neurone |
| | VSNARE | | |
| Tetanus | cellubrevin a VSNARE | ? | all cells |
| botulinum | SNAP2 a SNAP | near C | neurone |
| neurotoxin A | | terminus | |
| botulinum | synaptobrevin a | Q76F77 | neurone |
| neurotoxin B | VSNARE | | |
| botulinum | syntaxin a | near C | neurone |
| neurotoxin C | tSNARE | terminus | |
| botulinum | synaptobrevin | K59L60 | neurone |
| neurotoxin D | VSNARE | | |
| botulinum | SNAP25 a VSNARE | ? | neurone |
| neurotoxin E | | | |
| botulinum | synaptobrevin | Q58K59 | neurone |
| neurotoxin F | VSNARE | | |
| botulinum | cellubrevin a VSNARE | ? | all cells |
| neurotoxin F | | | |

Target Aberrant Human Functionalities

Numerous human abnormalities arise from aberrant functionalities of particular cellular proteins. As such inhibitory agents are useful therapeutical agents.

Ion Channels

Ion channels govern the electric activity of the body. It is therefore no surprise that pathologies such as hypertension are treated with ion channel blockers, most of which are natural products. Once again in a similar fashion to that stated above, any human channel inserted in functional form in the bacterial membrane will compromise the membrane potential and with it cellular viability.

Proteases

There are many examples of proteases in the body that upon “excessive” function may cause severe abnormalities. One such example is angiotensin converting enzyme (ACE), a protease whose action results in the production of the peptide hormone angiotensin leading to vasoconstriction and elevated blood pressure Thus, it should be of no surprise that any compound that would inhibit the ACE protease is a vasodilator and anti hypertensive agent (e.g. Benazepril; Captopril; Cilazapril; Enalapril; Enalaprilat; Fosinopril; Lisinopril; Moexipril; Perindopril; Quinapril; Ramipril; Trandolapril). Numerous other examples exist in the body, each of which is a potential target of the present teachings.

Protein Kinases

A protein kinase is a kinase enzyme that modifies other proteins by chemically adding phosphate groups to them (phosphorylation). Phosphorylation usually results in a functional change of the target protein (substrate) by changing enzyme activity, cellular location, or association with other proteins. The human genome contains about 500 protein kinase genes and they constitute about 2% of all human genes. Protein kinases are also found in bacteria and plants. Up to 30% of all human proteins may be modified by kinase activity, and kinases are known to regulate the majority of cellular pathways, especially those involved in signal transduction. Deregulated kinase activity is a frequent cause of disease, in particular cancer, wherein kinases regulate many aspects that control cell growth, movement and death Inhibitors of protein kinases are expected to be efficient in the treatment of various human diseases [as known to date for Gleevec (imatinib) and Iressa (gefitinib)].

An important embodiment of the present teachings is to select the polypeptide-of-interest or to express the polypeptide-of-interest such that is causes growth retardation.

Accordingly, the present invention further provides for a method of identifying a polypeptide which is incompatible with cell growth or vitality, the method comprising:
(a) expressing the polypeptide in cells being xenogeneic to the polypeptide; and
(b) measuring growth of the cells expressing the polypeptide compared to growth of control cells of the same origin not expressing the polypeptide under identical conditions, wherein when growth of the cells expressing the polypeptide is retarded compared to the control cells, the polypeptide is considered incompatible with cell growth or vitality.

Reduced growth or vitality refers to at least 20%, 30%, 40%, 50%, 60%, 70%, 90% less growth as compared to control cells (identical cells not expressing the polypeptide-of-interest) grown under identical growth conditions. It will be appreciated that measures are taken to provide for a moderate reduction in growth/vitality that is still amenable to reversion. That is, upon contact with an inhibitor, cell growth is resumed or increased at least in part. Further below are means to ensure such a moderate effect.

Methods of monitoring cell growth are well known in the art. For instance, mammalian cell growth/vitality can be assayed using any of the following exemplary methods: the MTT test which is based on the selective ability of living cells to reduce the yellow salt MTT (3-(4,5-dimethylthiazolyl-2)-2,5-diphenyltetrazolium bromide) (Sigma, Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; the Senescence associated-β-galactosidase assay (Dimri G P, Lee X, et al. 1995. A biomarker that identifies senescent human cells in culture and in aging skin in vivo. Proc Natl Acad Sci USA 92:9363-9367); alternatively or additionally, bacterial cell growth can be assayed by simple optical density or colony counting. Alternatively, spectrofluorometric measurements can be done. For example, cell permeant cyanine nucleic acid stains such as the SYTO™ are used for simple detection of the presence of bacteria, yeast, mammalian cells and other nucleic acid-containing cells. The SYTO dyes are essentially nonfluorescent except when bound to nucleic acids, where they become highly fluorescent, often with quantum yields exceeding 0.5. Consequently, it is usually not necessary to remove unbound stains before analysis. SYTO dyes are available with blue, green, orange or red fluorescence. The SYTO dyes rapidly penetrate the membranes of almost all cells, including bacteria and yeast. The various cell types can often be identified by their characteristic morphology or, in the case of flow cytometric applications, by their light-scattering properties.

The BacLight™ Green and BacLight™ Red bacterial stains are fluorescent, non-nucleic acid labeling reagents for detecting and monitoring bacteria. Bacteria stained with the BacLight™ Green and BacLight™ Red bacterial stains exhibit bright green (excitation/emission maxima ˜480/516 nm) and red (excitation/emission maxima ˜480/516 nm) fluorescence, respectively, and can be resolved simultaneously using the appropriate flow cytometry channels. Although these dyes were specifically chosen for flow cytometry applications, bacteria stained with these BacLight reagents can also be visualized by fluorescence microscopy with only minor, if any, adjustments in the staining concentrations. Furthermore, the BacLight bacterial staining patterns are compatible with formaldehyde or alcohol fixation methods.

Additionally or alternatively Kleymann and Werling J. Niomol. Screen. October 2004 vol. 9(7):578-587 describe methods for mammalian and bacterial cell enumeration and is hereby incorporated by reference in its entirety.

Cells suitable as host systems according to the present teachings, include, but are not limited to, isolated, cultured prokaryotic cells or eukaryotic cells, which are amenable to genetic transformation.

The use of microorganisms such as prokaryotic microorganisms may prove advantageous especially in the forced evolution embodiment because of the relatively short doubling time which positively affects in vitro evolution processes.

The term “microorganism” refers to any type of unicellular organism such as bacteria, protozoa and fungi (including yeast).

Examples of suitable eukaryotic cells include, but are not limited to, yeast, insect, fungi, plant and mammalian (e.g., human) cells.

Examples of prokaryotic microorganisms include, but are not limited to, bacteria and Archaea.

As mentioned hereinabove, a variety of prokaryotic or eukaryotic cells can be used as host-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptide-of-interest.

According to a specific embodiment the bacteria is Gram positive.

According to another specific embodiment, the bacteria is Gram negative bacteria.

Examples of bacterial strains which are amenable to genetic transformation include, but are not limited to, DH5a, Able C, TG-1, Sure2, DM-1, DB3.1, Topp-10, NovaBlue, Fusion Blue and Stb12.

Examples of bacterial constructs include the pET series of E. coli expression vectors [Studier et al. (1990) Methods in Enzymol. 185:60-89), as further described hereinbelow.

Escherichia coli may be advantageously used as a host since the ease of genetic manipulation is unrivaled by any other organism. It is suggested to express the polypeptide-of-interest using several expression systems such as the PET system employing the LacZ controllable promoter controlling the expression of the T7 polymerase [Moffatt et al. 1988 J. Bacteriol. 170(5):2095-21051. The T7 promoter may also drive a downstream antibiotic resistance gene so that the bi-cistronic message will ensure that the promoter does not get mutated under stress. The use of a multi-copy plasmid reduces the probability of resistance through mutations of the nucleic acid sequence encoding the polypeptide-of-interest. The experiments described below, necessitate tight control over the expression levels. If it found that even in the absence of an inducer, growth impairment is obtained, further measures to restrict expression can be achieved. For example in the PET systems it is possible to make use of the PlysE system which express a repressor of the T7 polymerase [Moffatt et al. 1988 J. Bacteriol. 170(5):2095-2105]. Alternatively, a different expression system can be used, such as the system that relies on the arabinose promoter [Stoner et al. 1983 J. Mol. Biol. 171(4):369-381] of lac promoter. Such a system may offer a broader range of expression levels, whereas the PET system, may be limited to the higher end of the expression spectrum.

An alternative approach is to conduct the expression of the polypeptide agent in the actinomycete Streptomyces lividans. Actinomycetes are by far the biggest producers of secondary metabolites known to date. Therefore they represent a rich source of genetic diversity from which to select agents that might inhibit the activity of the harmful agent. Unfortunately, heterologous expression of actinomycete proteins employing endogenous promoters in Escherichia coli is limited by transcriptional and translational barriers. Therefore it is suggested to express the polypeptide-of-interest in Streptomyces lividans, utilizing one of a range of widely available vectors (see e.g. the plasmid pCJR29 system Rowe 1998 Gene 216(1):215-223) or plasmids using the thiostrepton inducible promoter (tip) or the pristinamycin resistance promoter (ptr) which are activated by, respectively, addition of the antibiotic thiostrepton in sub lethal concentration, or nutritional downshift,

Rescue mechanisms (in addition to mutagens) include of libraries of from other/donor actinomycete species and are constructed in a low copy number plasmid, a derivative of SCP2* called pCJR29 [Rowe supra] and introduced into the Streptomyces lividans bearing the toxic functionality, to test for the ability of this cloned DNA to exert a protective effect.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art can also be used by some embodiments of the invention.

As mentioned, the polypeptide is expressed such that it causes growth retardation of the cells when expressed therein.

Methods of monitoring cell growth or growth arrest are well known in the art of cell biology and microbiology and described supra.

When needed the effect of the polypeptide-of-interest on cell growth is carefully controlled. According to one embodiment, the expression of the polypeptide is controlled using an inducible promoter. Inducible promoters suitable for use in accordance with some embodiments of the invention include for example the tetracycline-inducible promoter (Zabala M, et al., Cancer Res. 2004, 64(8): 2799-804) used in mammalian cells; the IPTG in the PET system in the case of expression in Escherichia coli; and thiostrepton or pristinamycin when expressing in Streptomyces lividans. It is possible to grow the cells in a medium without any induction of the polypeptide-of-interest. Subsequently transfer to solid medium which contains the inducer would result in the over expression of the harmful agent and the onslaught of external stress.

Yet alternatively or additionally, the cells expressing the polypeptide-of-interest are grown in the presence of a known inhibitor thereof (i.e., antidote). The cells are grown in the presence of the antidote and then plated on solid medium (in the case of bacterial cells) which does not contain the antidote. In this experiment the polypeptide-of-interest can be continuously expressed since its deleterious affects are negated.

Once a well controlled expression of the polypeptide-of-interest which results in adequate effect on cell growth is achieved, the cells are either subjected to an exogenous (heterologous) test agent or allowed to synthesize an endogenous inhibitor of the polypeptide-of-interest.

According to the first option, the cells (e.g., induced cells if necessary) are contacted with a test agent.

As used herein, the term “test agent” refers to a molecule(s) or a condition putatively capable of reversing or partially reversing the growth retardation effect of the polypeptide-of-interest.

Examples of molecules which can be utilized as agents according to the present invention include, but are not limited to, nucleic acids, e.g., polynucleotides, ribozymes, microRNAs, siRNAs and antisense molecules (including without limitation RNA, DNA, RNA/DNA hybrids, peptide nucleic acids, and polynucleotide analogs having altered backbone and/or bass structures or other chemical modifications); In this case, the nucleic acid agents are either contacted as naked DNA/RNA with the cells or form a part of a nucleic acid expression construct or library which are used along with transformation/transfection or infection protocols.

Other examples of molecules which can be utilized as agents according to the present invention include, but are not limited to, peptides, polypeptides, carbohydrates, lipids and “small molecule” drug candidates. “Small molecules” can be, for example, naturally occurring compounds (e.g., compounds derived from plant extracts, microbial broths, and the like) or synthetic organic or organometallic compounds having molecular weights of less than about 10,000 daltons, preferably less than about 5,000 daltons, and most preferably less than about 1,500 daltons.

Examples of conditions suitable for use as agents according to the present invention include, but are not limited to culturing conditions, such as, for example, temperature, humidity, atmospheric pressure, gas concentrations, growth media, contact surfaces, and the presence or absence of other cells in a culture.

According to the second option, the cells are subjected to a forced evolution process. There are two methods by which microorganisms can acquire resistance to a particular harmful agent: (i) Vertical evolution and (ii) Horizontal evolution.

Vertical evolution: Darwinian evolution is driven by principles of natural selection. In other words, random mutations that occur in an organism may further its chances of survival. Simple mathematics maintains that for any given population, several mutations should arise in a matter of hours (the mutation rate is roughly 10⁻⁸ and the population densities are >10⁹). Clearly the mutation rate can be enhanced through the use of mutagens (e.g., ethylmethylsulfonate, ethylnitrosourea or chemotherapeutic agents known in the art, such as adriamycin and the like).

Horizontal evolution: in this method, resistance is achieved through the acquisition of resistance factors from another organism. In this way organisms can rely on a far greater genetic background to confront any harmful agents.

Thus, in the forced evolution approach described herein tolerance to the growth Inhibitory effect of the polypeptide-of-interest is achieved via the production of a new small chemical.

Regardless of the method employed, the cells are monitored for a relief of the growth retardation (caused by the expression of the polypeptide-of-interest).

As used herein “a relief of the growth retardation” refers to at least an alleviation in the growth phenotype (e.g., kinetics) of the host cell expressing the polypeptide-of-interest to a complete reversion in growth phenotype towards that of a control cell (i.e., cell of identical origin not expressing the polypeptide-of-interest).

Once a test agent that causes a relief of said growth retardation is identified (either being endogenously synthesized by the host cell or exogenously contacted with the host cell) it is isolated.

In the case of an endogenously synthesized molecule, it is possible to distinguish it through a simple filtration assay of the conditioned media of resistant cells. In other words, take the media from cells (In case of secreted molecule) or cell lysates of cells that have developed resistance. Filter it through pore of a given size (e.g. <5,000 Da) and check if the filtrate can confer resistance to sensitive bacteria. Subsequently one can fractionate the filtrate and identify the active compound.

Thus in order to validate the effect of either an endogenously synthesized molecule or an exogenous test agent, the present invention further envisages contacting the cells expressing the polypeptide-of-interest (e.g., an independent sample of the same cells) with the isolated test agent or molecule to identify the relief of the growth retardation.

A test agent or molecule causing the relief of the growth retardation is synthesized and may be developed as a candidate lead compound in research or clinical applications.

Although the present invention can be practiced with a single cell sample or only several cell samples, it may be advantageously used for high throughput screening of agents using a plurality of cells to simultaneously screen a plurality of agents.

In such a large scale throughput screening approach the agent may be part of a library, such as a small molecule library or an expression library. Chemical libraries are available, for example, from chemical companies including Merck, Glaxo, Novartis, and Bristol Meyers Squib. Optionally, libraries of natural compounds in the form of bacterial, fungal, plant and animal extracts, which are available from, for example, Pan Laboratories or Mycosearch or are readily producible by methods known in the art can also be utilized by the present invention.

When performing large-scale screening, the cell population that is subjected to individual agents can be compartmentalized so as to facilitate identification of abnormal phenotype reversal. This may be effected by alliquoting the cell population into flat glass-bottom multiwell plates at a pre-calibrated density which allows the growth of just one or two clones per well.

In the step of screening a manual procedure can be followed, although automated screening using robots, such as multiwell attachment for the DeltaVision microscope, Cellomics automated microscope, are preferred.

Once identified, agents capable of at least partially reversing the growth phenotype as described above are recovered. If the agent is a polynucleotide or a polynucleotide expression product, cells are isolated and propagated and are used for isolating the polynucleotides agents, by, for example, PCR amplification, as discussed above.

The retrieved agents are further analyzed for their exact mechanism of action and adjusted for optimal effect, using various biochemical and cell-biology methods.

Eventually, distinguishing which of the agent isolated is a potential a lead compound can be accomplished by testing the effect of the agent in pharmacological models of various diseases. Agents that affect disease progression or onset, constitute leads for drug development.

Such agents can be applied for treatment of many pathological states such as cancer, metabolic disorders such as, diabetes and obesity, cardiopulmonary diseases, inflammatory diseases, viral infections, bacterial infections and other known syndromes and diseases.

The present teachings further contemplate kits which comprise the isolated cells expressing the polypeptide-of-interest (e.g., bacterial cells expressing the M2 polypeptide) for use in screening settings such as described above and are provided along with instructions for use.

It is expected that during the life of a patent maturing from this application many relevant host systems and screening settings will be developed and the scope of the terms described herein is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### A Rapid, Accurate and Economic Method to Identify Channel Inhibitors Demonstrated on Influenza M2

The outline of the strategy includes the introduction of ion channels from various viruses by genetic engineering into bacteria. Expression of the channels so as to result in growth retardation; and enhancement of the innate ability of the bacteria to respond to the viral stress; and monitoring the bacteria for the emergence of resistance.

### Materials and Experimental Procedures

Chemicals—Amantadine, Rimantadine and the HIS-Tag antibody kit were all purchased from Sigma-Aldrich laboratories, Israel. Isopropyl,6-D-thiogalactopyranoside (IPTG) was purchased from BiochemikaFluka (Buchs, Switzerland) and distributed into aliquots stored on −20° c. The pMal-p2x vector was purchased from New England Biolabs (Ipswich, Mass.). pET-22b(+) plasmid and Tuner (DE3) cells were purchased from Novagen (Gibbstown, N.J.). DH10B bacteria cells were purchased from Invitrogen Israel. Western gel kit (chemicals and a dual color marker) was purchased from BioRad Israel.

Plasmid and Bacterial Strain Design—The Singapore M2 sequence was synthesized by GenScript (Piscataway, N.J.). This wild-type construct was designed according to the Singapore H2N2 isolate, M2 sequence (6). The gene was flanked by the N col and HindIII restriction sites, in the pUC57 plasmid. The sequence was transferred with the former 2 restriction sites to the pMal-p2x plasmid via XmnI and XbaI restriction sites, in frame with the MalE protein—following a poly Asn site. Different bacterial cell types were screened in this assay, as hosts for the above plasmid. As described in the results section, reproducible results were achieved with the DH10B and Tuner (DE3) cells. All other forms of the M2 proteins were obtained from mutations of the Singapore wild-type strain with the Quick multiMuatagenesis kit from Stratagene (La Jolla, Calif.).

Cells Growth—Cells bearing or lacking (as a reference) the ion channel genes were incubated overnight from glycerol stocks with 100 μLg/ml Ampicillin LB. Thereafter, the growth culture was diluted 100 fold and the bacteria were grown until their O.D.600 reached 0.07-0.15. Cells were then divided into the wells of a 96 or 48 well flat bottomed plates containing the different treatments. The growth volume in the case of the 48 or 96 well plates was 500 μI and 100 μL, respectively. IPTG was added to the cells pool immediately after the LB media's O.D.600 reached ca. 0.15 The 48/96 well plates were incubated for 16 hours at 30° C. in a Synergy 2 multi-detection micro-plate reader from Biotek (Winooski, Vt.) at a constant high shaking rate. O.D.600 readings were recorded every each 30 min.

Western Blotting—Bacteria were grown as described above, with 50 μM IPTG induction, in the presence or absence of 100 μM Rimantadine for the indicated times. From every time point, 0.5 ml were taken from the growth culture and harvested by centrifugation. The pellet was washed in PBS buffer and then resuspended in the SDS sample buffer (2% SDS) at pH=6.8, containing 10% DTT, followed by heating to 60° C. for 20 min and intensive tip sonication (vibracell by sonics, Newtown, Conn.).

The sample was then loaded onto a 10% polyacrylamide gel and electrophoresed for 35 min under 30 mA. The gel was then blotted onto a nitrocellulose membrane and visualization of the Singapore wild-type M2-MaIE chimera was possible via blotting with an anti His-Tag antibody kit from Sigma-Aldrich laboratories, Israel.

Inhibitory constant derivation—Monod coefficients (Kₛ) were derived by measuring the dose response effect of amantadine and rimantadine upon the maximal growth rate of the host bacteria.

The resulting data were non-linearly fit according to the Monod equation relating the 150 kDa growth rate (R) to the drug concentration:

$$R = \frac{{R}_{max} [ drug ]}{{K}_{s} + [ drug ]}$$

Results

The objective of this study was to develop an assay to measure channel activity and the inhibition thereof. The assay must be both accurate to be used in detailed analyses and simple such that is can be used in high-throughput screening. Based on these two considerations a cell-based assay was designed such that the channel protein is expressed in bacteria resulting in growth retardation. The effects of channel blockers can then be assayed by their ability to relieve the aforementioned growth retardation. The channel studied is the M2 H+ channel, a critical component of the viral life cycle.

Protein Expression

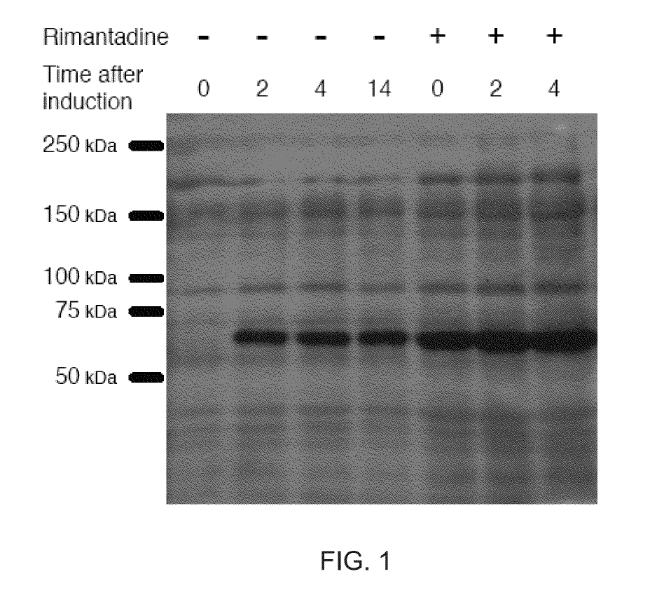

The first step of the assay was to ensure proper expression and reconstitution of the channel. In order to maintain successful incorporation into the bacterial inner membrane. The M2 protein was fused to the C-terminus of the maltose binding protein. FIG. 1 shows the profile of channel expression in the bacteria as a function of time and the presence of an inhibitor. Sodium dodecyl sulfate polyacrylamide gel electrophoresis of whole cell lysates exhibits a band at the calculated molecular weight of the chimeric protein (60 kDa). No protein is seen in the absence of induction, while two hours or more post induction, expression is clearly visible. Interestingly, the expression of the protein is enhanced when the bacteria are grown in the presence of the anti-flu drug rimantadine. The reasons for this finding are elaborated below.

Bacterial Growth

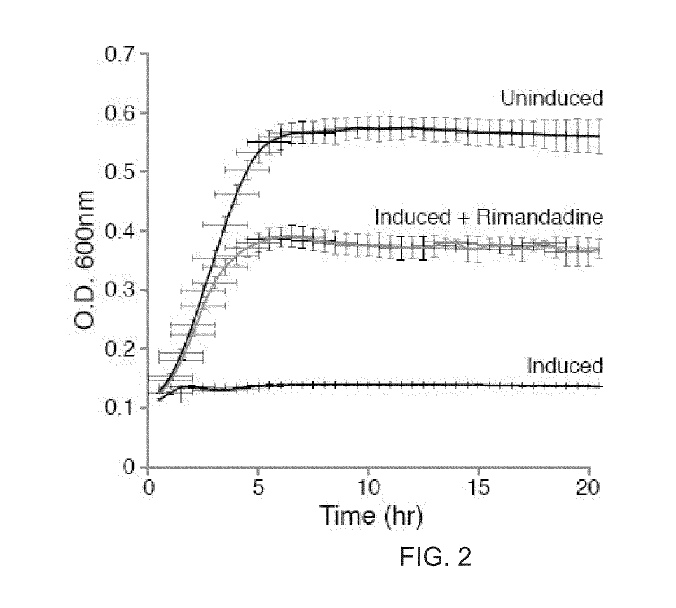

Following evidence that the M2 chimera is expressed in bacterial cells, the protein's effect upon growth rate of the bacteria was examined. As shown in FIG. 2, bacteria that express the ion channel are virtually incapable of growth as compared to bacteria that do not express the M2 chimera.

However, it was essential to establish that the growth impairment is specific to the proteins channel activity and not just a general deleterious effect due to overexpression of a heterologous membrane protein [R Grisshammer and C G Tate. Overexpression of integral membrane proteins for structural studies. Q Rev Biophys, 28(3):315-422, 1995.].

This was achieved by demonstrating that a specific channel blocker of the protein-the anti flu agent rimantadine can significantly alleviate the growth retardation (FIG. 2). Taken together, it is possible to conclude that the viral ion channel permeabilizes the bacterial membrane, resulting in growth retardation, that can be reversed by the activity of a specific channel blocker.

Channel Strain Effect

After demonstrating that the activity of the viral channel can be specifically studied in the bacterial membrane, the effects of channels from various viral strains were examined. Specifically, the following five different Influenza H⁺ channels, each from a different viral strain were examined: (i) Singapore; (ii) Rostock; (iii) Singapore with a mutation of S31N; (iv) Swine flu; and (v) BM2 from Influenza B. The mutation of Ser31 to Asn is known to render the channel insensitive to aminoadamantanes [Y Suzuki, R Saito, H Zaraket, C Dapat, I Caperig-Dapat, and H Suzuki. Rapid and specific detection of amantadine resistant influenza A viruses with a Ser31Asn mutation by the cycling probe method. J Clin Microbial, 48(1):57-63, 2010].

Furthermore, swine flu is known to contain the S31N mutation and hence is assumed to be resistant to aminoadamantanes [L H Pinto, L J Holsinger, and R A Lamb. Influenza virus M2 protein has ion channel activity. Cell, 69(3):517-28, 1992.]. Finally, the BM2 channel from influenza B is known to be resistant to aminoadamantanes as well [J A Mould, R G Paterson, M Takeda, Y Ohigashi, P Venkataraman, R A Lamb, and L H Pinto. Influenza B virus BM2 protein has ion channel activity that conducts protons across membranes. Dev Cell, 5(1): 175-84, 2003.].

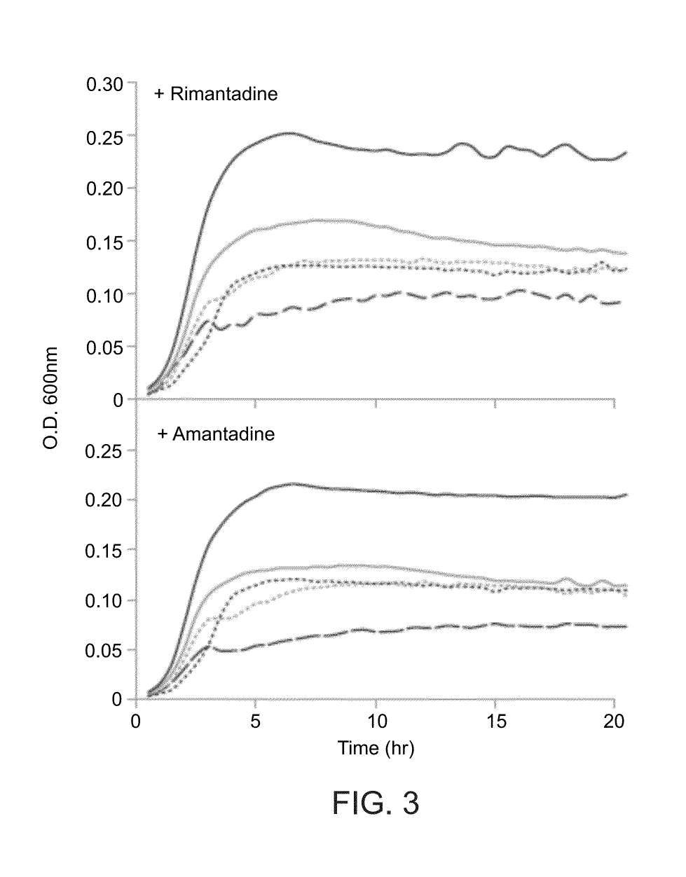

As shown in FIG. 3, the different channels indeed exhibit different sensitivities to the aminoadamantane channel blockers. The growth inhibition by the channel from the Singapore strain is substantially relieved upon addition of either rimantadine or amantadine. In contrast, the effect of the drugs upon the growth retardation by channels from aminoadamantane resistant viruses was significantly smaller. For example, only a single mutation—S31N in the expressed channel diminished the ability of aminoadamantanes to relieve growth inhibition by more than 50% (compare solid black line versus dotted gray line in FIG. 3). Similarly, as expected aminoadamantanes exhibit poor growth retardation relief for bacteria that express the BM2 from influenza B that is known to be refractive to aminoadamantanes.

However, it is important to note that the above assay is sufficiently sensitive to detect the marginal aminoadamantane sensitivity of the resistant channels such as the Singapore S31N mutant. The assay is able to detect even the reduced levels of channel blocking which is important for high-throughput screening. The reason being that such screens would be able to identify even poorly blocking compounds, the activity of which can then be enhanced by further optimization.

Drug Effect

Having established that the assay can quantitatively detect differences in sensitivity towards aminoadamantane of the different channels, the present inventor examined if differences between various drugs can be observed as well. The growth retardation relief effects of the only two aminoadamantanes that are approved for prophylactic use were compared: rimantadine and amantadine. Detailed comparison between the top and bottom panels of FIG. 3 does indeed show that that activity of rimantadine is more pronounced than amantadine [P E Aldrich, E C Hermann, W E Meier, M Paulshock, W W Prichard, J A Snyder, and J C Watts. Antiviral agents. Structure-activity relationships of compounds related to adamantanamine. J Med Chem, 14(6):535-43, 1971.; X Jing, C Ma, Y Ohigashi, F A Oliveira, T S Jardetzky, L H Pinto, and R A Lamb. Functional studies indicate amantadine binds to the pore of the influenza A virus M2 proton-selective ion channel. Proc Natl Acad Sci USA, 105(31):10967-72, 2008; ; V Balannik, V Carnevale, G Fiorin, B G Levine, R A Lamb, M L Klein, W F Degrado, and L H Pinto. Biochemistry, 2009.). Specifically, one can see that regardless of the channel that is expressed the bacteria were able to grow to higher O.D.600 when rimantadine was added to the media in comparison to amantadine.

The different activities of the two drugs reflects the known advantage of rimantadine over amantadine in channel blocking that is mirrored in the present assay.

Kₛ Measurements

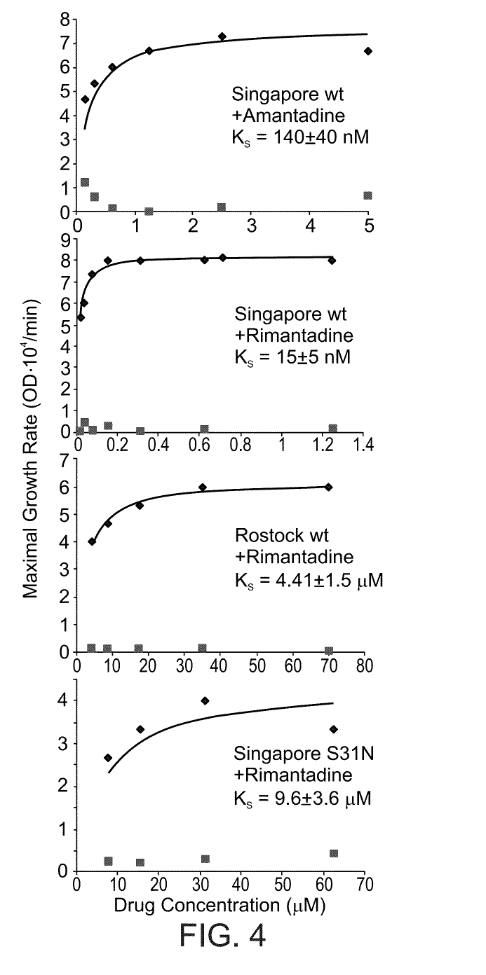

The final demonstration of the quantitative nature of the assay is the derivation of Kₛ values for various drug and channels. Here the Kₛ values were derived by measuring the dose response effect of amantadine and rimantadine upon the maximal growth rate of the host bacteria. As seen in FIG. 4, irrespective of the channel variant, or the drug, the measured data fit remarkably well to the Monod equation. The derived K; for the Singapore wild-type strain for amantadine and rimantadine are 140 nM and 15 nM, respectively.

It is difficult to compare the results to the isochronic apparent inhibitory binding constant values reported by Lamb and co-workers [Jing, C Ma, Y Ohigashi, F A Oliveira, T S Jardetzky, L H Pinto, and R A Lamb. Functional studies indicate amantadine binds to the pore of the influenza A virus M2 proton-selective ion channel. Proc Natl Acad Sci USA, 105(31):10967-72, 2008; C Wang, K Takeuchi, L H Pinto, and R A Lamb. Ion channel activity of influenza A virus M2 protein: characterization of the amantadine block. J Viral, 67(9):5585-94, 1993.]. The reasons are that isochronic measurements by their very nature depend on the time when the measurements are taken. Hence, direct comparison to the present procedure is not possible. In general one may note that the present study yields significantly higher activities of aminoadamantanes relative to the isochronic approach. For example the Kₛ of amantadine in the wild-type Singapore channel is 140 nM (FIG. 4) in comparison to16 μM in the isochronic study ([Jing, C Ma, Y Ohigashi, F A Oliveira, T S Jardetzky, L H Pinto, and R A Lamb. Functional studies indicate amantadine binds to the pore of the influenza A virus M2 proton-selective ion channel. Proc Natl Acad Sci USA, 105(31):10967-72, 2008). Furthermore, the decrease in the S31N mutant sensitivity towards the drug in the isochronic study is an order—16 to 200 JLM ([Jing, C Ma, Y Ohigashi, F A Oliveira, T S Jardetzky, L H Pinto, and R A Lamb. Functional studies indicate amantadine binds to the pore of the influenza A virus M2 proton-selective ion channel. Proc Natl Acad Sci USA, 105(31):10967-72, 2008). In the present approach a much higher dynamic range is obtained (this time to rimantadine) 15 nm to 9.6 μM.

The last issue to consider is the dynamic range of the present assay. As seen in FIG. 4, the present cell based assay yields detailed Kₛ measurements in a span of three orders of magnitude—from micro to nano-molar. This aspect is critical to allow the assay to be used in high throughput screening since it ensures that one would be able to detect leads that are both highly efficacious along side marginally active compounds.

### Example 2

### Hepatitis C p7 and HIV Vpu Polypeptides

Materials and Methods

The p7 and Vpu genes were also constructed by GenScript Corporation. The viral channel proteins p7 (e.g., taken from GenBank Accession ACJ37217.1) and Vpu (GenBank Accession AAF35359.1) were expressed in a number of vector/bacterial host combinations:
pET-22b(+) in Origami B.
pET-22b(+) in HMS 174.
pMAL-p2X in DH5-alpha.
pMAL-p2X in DH10B.
pMAL-p2X in NT236.
pMAL-p2X in EP432.
pMAL-p2X in KNabc.

The growth conditions are similar to the influenza M2.

Results

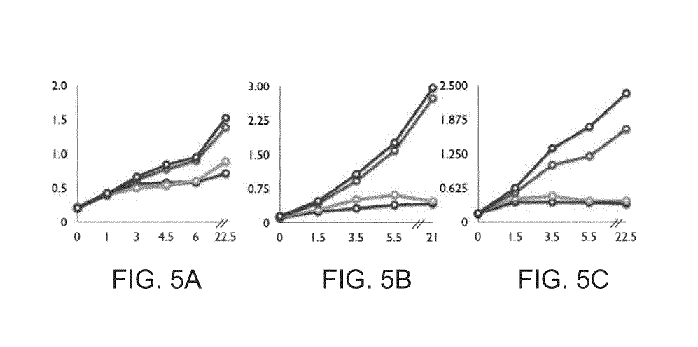

Bacterial growth in all of the combinations listed above was inhibited to various extents. Specifically, p7 and Vpu were expressed in pMAL-p2X in the E. coli strain KNabc for two reasons: (i) this strain lacks the three major E. coli sodium-proton antiporters, and therefore cannot extrude sodium from the cell. As such it is highly sensitive to the presence of sodium in the growth medium (see FIGS. 5A-C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited in the Document

[1] R H Baltz and T J Hosted. Molecular genetic methods for improving secondary metabolite production in actinomycetes. Trends Biotechnol, 14(7):245-249, 1996.
[2] T Betakova, M V Nermut, and A J Hay. The NB protein is an integral component of the membrane of Influenza B virus. J Gen Virol, 77 (Pt 11):2689-2694, 1996.
[3] M Bui, G Whittaker, and A Helenius. Effect of ml protein and low ph on nuclear transport of Influenza virus ribonucleoproteins. J Virol, 70:8391-8401, 1996.
[4] M J Coady, N G Daniel, E Tiganos, B Allain, J Friborg, J Y Lapointe, and E A Cohen. Effects of vpu expression on Xenopus oocyte membrane conductance. Virology, 2440:39-49, 1998.
[5] E A Cohen, E F Terwilliger, J G Sodroski, and W A Haseltine. Identification of a protein encoded by the vpu gene of HIV1. Nature, 334(6182):532-534, 1988.
[6] P L Collins and G Mottet. Oligomerization and posttranslational processing of glycoprotein G of human respiratory syncytial virus: altered 0 glycosylation in the presence of brefeldin A. J Gen Virol, 73(4):849-863, 1992.
[7] J R Doedens and K Kirkegaard Inhibition of cellular protein secretion by poliovirus proteins 2B and 3A. EMBO J, 14(5):894-907, 1995.
[8] J Friborg, A Ladha, H Gottlinger, W A Haseltine, and E A Cohen. Functional analysis of the phosphorylation sites on the human immunodeficiency virus type 1 Vpu protein. J Acqu Immu Defi Synd, 8(1):10-22, 1995.
[9] K Geering. Na, KATPase. Curr Opin Nephr Hyperten, 6(5):434-439, 1997.
[10] H G Gottlinger, T Dorfman, E A Cohen, and W A Haseltine. Vpu protein of human immunodeficiency virus type 1 enhances the release of capsids produced by gag gene constructs of widely divergent retroviruses. Proc Nat Acad Sci USA, 90(15):7381-7385, 1993.
[11] R Guinea and L Carrasco. Influenza virus M2 protein modifies membrane permeability in E. coli cells. FEBS Lett, 343(3):242-246, 1994.
[12] A Helenius. Unpacking the incoming Influenza virus. Cell, 69(4):577-8, 1992.
[13] S W Hiebert, R G Paterson, and R A Lamb. Identification and predicted sequence of a previously unrecognized small hydrophobic protein, SH, of the paramyxovirus Simian virus 5. J Virol, 55(3):744-51, 1985.
[14] S Hongo, K Sugawara, Y Muraki, F Kitame, and K Nakamura. Characterization of a second protein (CM2) encoded by RNA segment 6 of Influenza C virus. J Virol, 71(4):2786-2792, 1997.
[15] S Hongo, K Sugawara, H Nishimura, Y Muraki, F Kitame, and K Nakamura. Identification of a second protein encoded by Influenza C virus RNA segment 6. J Gen Virol, 75(12):3503-3510, 1994.
[16] T Klimkait, K Strebel, M D Hoggan, M A Martin, and J M Orenstein. The human immunodeficiency virus type 1 specific protein vpu is required for efficient virus maturation and release. J Virol, 64(2):621-629, 1990.
[17] M Kordel, F Schuller, and H G Sahl. Interaction of the pore forming peptide antibiotics Pep 5, nisin and subtilin with nonenergized liposomes. Febs Lett, 244(1):99-102, 1989.
[18] F A Kovacs and T A Cross. Transmembrane four helix bundle of InfluenzaA M2 protein channel: structural implications from helix tilt and orientation. Biophys J, 73(5):2511-2517, 1997.
[19] F A Kovacs, J K Denny, Z Song, J R Quine, and T A Cross. Helix tilt of the M2 transmembrane peptide from Influenza A virus: An intrinsic property. J Mol Biol, 295(1):117-125, 2000.
[20] A Kukol, P D Adams, L M Rice, A T Brunger, and I T Arkin. Experimentally based orientational refinement of membrane protein models: A structure for the Influenza A M2 H+ channel. J Mol Biol, 286:951-962, 1999.
[21] A Kukol and I T Arkin. Structure of the HIV1 Vpu transmembrane complex determined by site specific FTIR dichroism and global molecular dynamics searching. Biophys J, 77:1594-1601, 1999.
[22] A Kukol and I T Arkin. Structure of the Influenza C CM2 protein transmembrane domain obtained by sites specific infrared dichroism and global molecular dynamics searching. J Biol Chem, 78:55-69, 2000.
[23] S Kurtz, G Luo, K M Hahnenberger, C Brooks, O Gecha, K Ingalls, K Numata, and M Krystal. Growth impairment resulting from expression of Influenza virus M2 protein in Saccharomyces cerevisiae: identification of a novel inhibitor of Influenza virus. Antimicrob Agents Chemother, 39(10):2204-2209, 1995.
[24] R A Lamb and L H Pinto. Do Vpu and Vpr of human immunodeficiency virus type 1 and NB of Influenza B virus have ion channel activities in the viral life cycles? Virology, 229(1): 1-11, 1997.
[25] F Maldarelli, R L Willey, and K Strebel. Human immunodeficiency virus type 1 Vpu protein is an oligomeric type I integral membrane protein. J Virol, 67(8):5056-5061, 1993.
[26] F M Marassi and S J Opella. NMR structural studies of membrane proteins. Curr Opin Struc Biol, 8(5):640-648, 1998.
[27] I Marczinovits, I Boros, F El Jarrah, G Fust, and J Molnar. Expression in Escherichia coli and in vitro processing of HIV1 p24 fusion protein. J Biotechnol, 31(2):225-232, 1993.
[28] J F Martin and P Liras. Organization and expression of genes involved in the biosynthesis of antibiotics and other secondary metabolites. Annu Rev Microbiol, 43:173-206, 1989.
[29] C G Miyada, D E Sheppard, and G Wilcox. Five mutations in the promoter region of the araBAD operon of Escherichia coli B/r. J Bacteriol, 156(2):765-772, 1983.
[30] B A Moffatt and F W Studier. Entry of bacteriophage T7 DNA into the cell and escape from host restriction. J Bacteriol, 170(5):2095-2105, 1988.
[31] Z Oren and Y Shai. A class of highly potent antibacterial peptides derived from pardaxin, a poreforming peptide isolated from Moses sole fish Pardachirus marmoratus. Eur J Biochem, 237(1):303-310, 1996.
[32] A K Patick and K E Potts. Protease inhibitors as antiviral agents. Clin Microbiol

Rev, 11(4):614-627, 1998.
[33] A Pekosz and R A Lamb. The CM2 protein of Influenza C virus is an oligomeric integral membrane glycoprotein structurally analogous to Influenza A virus M2 and Influenza B virus NB proteins. Virology, 237(2):439-451, 1997.
[34] L H Pinto, L J Holsinger, and R A Lamb. Influenza virus M2 protein has ion channel activity. Cell, 69(3):517-528, 1992.
[35] B Roux and R MacKinnon The cavity and pore helices in the KcsA K+ channel: Electrostatic stabilization of monovalent cations. Science, 285(5424):100-102, 1999.
[36] C J Rowe, J Cortes, S Gaisser, J Staunton, and P F Leadlay. Construction of new vectors for high level expression in actinomycetes. Gene, 216(1):215-223, 1998.
[37] D Salom, B R Hill, J D Lear, and W F DeGrado. pH dependent tetramerization and amantadine binding of the transmembrane helix of M2 from the Influenza A virus. Biochemistry us, 39(46):14160-14170, 2000.
[38] M A Sanz, L Perez, and L Carrasco. Semliki Forest virus 6K protein modifies membrane permeability after inducible expression in Escherichia coli cells. J Biol Chem, 269(16):12106-10, 1994.
[39] U Schubert, S Bour, A V Ferrer Montiel, M Montal, F Maldarelli, and K. The two biological activities of human immunodeficiency virus type 1 Vpu protein involve two separable structural domains. J Virol, 70(2):809-819, 1996.
[40] U Schubert and K Strebel. Differential activities of the human immunodeficiency virus type 1 encoded Vpu protein are regulated by phosphorylation and occur in different cellular compartments. J Virol, 68(4):2260-2271, 1994.
[41] V V Smirnov, E A Kiprianova, A D Garagulya, S E Esipov, and S A Dovjenko. Fluviols, bicyclic nitrogen rich antibiotics produced by Pseudomonas fluorescens. Fems Microbiol Lett, 153(2):357-361, 1997.
[42] C Stoner and R Schleif. The araE low affinity L arabinose transport promoter. Cloning, sequence, transcription start site and DNA binding sites of regulatory proteins. J Mol Biol, 171(4):369-381, 1983.
[43] K Strebel, T Klimkait, and M A Martin. A novel gene of HIV 1, vpu, and its 16 kilodalton product. Science, 241(4870):1221-1223, 1988.
[44] N A Sunstrom, L S Premkumar, A Premkumar, G Ewart, G B Cox, and P W Gage. Ion channels formed by NB, an InfluenzaB virus protein. J Membr Biol, 150(2):127-32, 1996.
[45] Q Tu, L H Pinto, G Luo, M A Shaughnessy, D Mullaney, S Kurtz, M Krystal, and R A Lamb. Characterization of inhibition of M2 ion channel activity by BL1743, an inhibitor of Influenza A virus. J Virol, 70(7):4246-4252, 1996.
[46] F Van Bambeke, M P MingeotLeclercq, A Schanck, R Brasseur, and P M Tulkens. Alterations in membrane permeability induced by aminoglycoside antibiotics: Studies on liposomes and cultured cells. Euro J Pharm Mol Pharm, 247(2):155-168, 1993.
[47] C Wang, R A Lamb, and L H Pinto. Activation of the M2 ion channel of Influenza virus: a role for the transmembrane domain histidine residue. Biophys J, 69(4):1363-1371, 1995.
[48] M A Williams and R A Lamb. Determination of the orientation of an integral membrane protein and sites of glycosylation by oligonucleotide directed mutagenesis: Influenza B virus NB glycoportein lacks a cleavable signal sequence and has an extracellular NH2terminal region. Mol Cell Biol, 6(12):4317-4328, 1986.
[49] V Wray, D Mertins, M Kiess, P Henklein, W Trowitzsch Kienast, and U. Solution structure of the cytoplasmic domain of the human CD4 glycoprotein by CD and 1H NMR spectroscopy: Implications for biological functions. Biochemistry, 37(23):8527-8538, 1998.
[50] S Y Zheng, J Strzalka, C Ma, S J Opella, B M Ocko, and Blasie J K. Structural study of the HIV1 accessary protein Vpu via synchrotron X ray reflectivity from langmuir monolayers. Biophys J, 78:945Pos, 2000.
[51] J A Mould, R G Paterson, M Takeda, Y Ohigashi, P Venkataraman, R A Lamb, L H Pinto. Influenza B virus BM2 protein has ion channel activity that conducts protons across membranes. Dev Cell, 5(1):175-84. 2003
[52] R G Paterson, M Takeda, Y Ohigashi, L H Pinto, R A Lamb. Influenza B virus BM2 protein is an oligomeric integral membrane protein expressed at the cell surface. Virology. 306(1):7-17, 2003

1. A method of identifying an inhibitor of a polypeptide-of-interest, the method comprising:
(a) expressing the polypeptide-of-interest in cells being xenogeneic to the polypeptide, wherein said polypeptide-of-interest is selected causing growth retardation of said cells when expressed therein;
(b) contacting said cells expressing said polypeptide-of-interest with a test agent;
(c) measuring growth of said cells following or concomitant with step (b), wherein a relief in said growth retardation is indicative that said test agent is an inhibitor of said polypeptide-of-interest.

2. The method of claim 1, further comprising synthesizing the test agent being the inhibitor of said polypeptide-of-interest.

3. A method of identifying an inhibitor of a polypeptide-of-interest, the method comprising:
(a) expressing the polypeptide-of-interest in cells being xenogeneic to the polypeptide, wherein said polypeptide-of-interest is selected causing growth retardation of said cells when expressed therein;
(b) culturing said cells expressing said polypeptide-of-interest under conditions which relieve said growth retardation, wherein said relief of said growth retardation is indicative of conditions that inhibit said polypeptide-of-interest.

4. A method of identifying a polypeptide which is incompatible with cell growth or vitality, the method comprising:
(a) expressing the polypeptide in cells being xenogeneic to the polypeptide; and
(b) measuring growth of said cells expressing the polypeptide compared to growth of control cells of the same origin not expressing the polypeptide under identical conditions, wherein when growth of said cells expressing the polypeptide is retarded compared to said control cells, the polypeptide is considered incompatible with cell growth or vitality.

5. The method of claim 3, wherein said conditions comprise a molecule endogenously synthesized by said cells.

6. The method of claim 5, further comprising synthesizing said molecule.

7. The method of claim 5, further comprising contacting said cells expressing said polypeptide-of-interest with said molecule to identify said relief of said growth retardation, thereby validating said inhibitory activity of said molecule.

8. The method of claim 1, wherein said polypeptide-of-interest is a recombinant peptide.

9. The method of claim 1, wherein a selection of said polypeptide of-interest causing growth retardation of said cell culture is performed according to the method of claim 1.

10. The method of claim 1, wherein said cells are microbial cells.

11. The method of claim 10, wherein said microbial cells are bacterial cells.

12. The method of claim 11, wherein said bacterial cells comprise Gram positive bacteria.

13. The method of claim 11, wherein said bacterial cells comprise Gram negative bacteria.

14. The method of claim 10, wherein said polypeptide is a human polypeptide.

15. The method of claim 1, wherein expressing the polypeptide comprises induced expression.

16. The method of claim 1, wherein said expressing is effected at least in part in a presence of a known inhibitor of said polypeptide so as to prevent death of said cells.

17. The method of claim 1, wherein the polypeptide is not a chimeric polypeptide.

18. The method of claim 1, wherein said polypeptide is a disease causing polypeptide.

19. The method of claim 1, wherein said polypeptide-of-interest is selected from the group consisting of an ion channel and a protease.

20. The method of claim 1, wherein said test agent comprises a nucleic acid sequence and wherein contacting refers to transforming said cells to express said nucleic acid sequence.

21. The method of claim 1, being effected in high throughput configuration.

22. The method of claim 21, wherein said test agent forms a part of a library.

23. The method of claim 18, wherein said disease causing polypeptide comprises a viral polypeptide.

24. The method of claim 23, wherein said viral polypeptide comprises an influenza polypeptide.

25. The method of claim 18, wherein said influenza polypeptide is M2.

26. An isolated bacterial cell expressing an influenza polypeptide.
